(19) United States
(12) Patent Application Publication
(10) Pub. No.: US 2004/0199091 A1
(43) Pub. Date: Oct. 7, 2004

(54) Testicular support device

(76) Inventor: Julie Vogelsang-Switzer, Fernie (CA)

Correspondence Address: GOWLING LAFLEUR HENDERSON LLP, SUITE 1400, 700 2ND ST. SW, CALGARY, AB, T2P 4V5, CA

(21) Appl. No.: 10/810,557

(22) Filed: Mar. 29, 2004

Related U.S. Application Data
(60) Provisional application No. 60/460,386, filed on Apr. 7, 2003.

(51) Int. Cl. A61F 5/00

(52) U.S. Cl. 602/1

(57) ABSTRACT

A tray device which goes over and between the legs for raising and supporting human testes during ultasound inspection, to assist in immobilizing same to improve ultrasonic imaging, adapted for use when the patient is in a supine position. A pair of spaced-apart downwardly curved members are provided, having a curvature approximating the curvature of a leg of a patient proximate the thigh area. Intermediate each of said curved members is a support member, which is of a narrow width to permit positioning of such support member beneath the testes of a male patient and between the legs of a patient to raise and support such testes to permit ultrasonic examination of such testes. The device may be a unitary one-piece construction, preferably of a moulded plastic , which also facilitates easy sterilization to permit re-use of the device on subsequesnt patients.

### FIELD OF THE INVENTION

This invention relates to a medical device, and in particular a device for supporting the testes of human males during ultrasound examinations.

### BACKGROUND OF THE INVENTION AND DESCRIPTION OF THE PRIOR ART

Scrotal and testicular examinations of human males by ultrasound techniques is frequently necessary for determination as to whether the testes of a human male are affected by disease or injury.

For example, injury to the testes of males frequently occurs due to sports-related activities, such as baseball, hockey, bicycle/biking, and karate. As well, examination of the testes of males is necessary where disease, such as testicular cancer, tumours, or the like is suspected.

Typically, due to potential harmful effects of x-rays on sperm in mature male testes, and the fact that x-ray imaging is typically an imaging modality used primarily for bone and hard structures (unless opaque substances such as barium are used, such as for enhanced imaging of stomachs, intestine, colon, and bowel, which is not applicable in the case of human testes), ultrasound inspection of the scrotum and the testes therein is the typical preferred means of inspecting testes for injury and/or disease.

Ultrasound inspection involves the administration of low-frequency sound pulses to human tissue, which reflected sound waves permit the production of an images of the tissue on a cathode ray or similar display monitor.

Testicular and scrotal examinations via ultrasound methods need be conducted not only on mature adult males, but also on juvenile males as well as young male children, particularly in cases where injury to the testes is suspected.

In order to conduct ultrasound examination of the testes/scrotum, it is necessary that the testes/scrotum be supported and immobilized to allow proper imaging on an ultrasound monitor. Movement of the testes/scrotum while imaging during an ultrasound examination is undesirable, as among other things it results in poor image display on an ultrasound monitor, and photographs/images obtained from such ultrasound inspection are blurred and unclear, making them useless for inspection by an attending physician.

The practice of ultrasound departments in most hospitals and out-patient clinics is to attempt to support and immobilize the scrotum/testes during an ultrasound examination by means of rolled-up towels, which are positioned between the patients legs and under the testes/scrotum while the patient is on his back on an examination table, thereby raising and supporting such testes during the ultrasound examination. According to such prior art method, towels are employed to raise, expose, and support the testes/scrotum of the male patient so as to allow an ultrasound probe to pulse sound waves into the scrotum/testes for the purpose of viewing such inspected tissue on an ultrasound display monitor.

The prior art method of immobilization , support and positioning of the scrotum/testes by means of towels is unsatisfactory for a number of reasons.

Firstly, it is difficult and time consuming to position the towels into the proper position to achieve the desired and sufficient raising and support of the testes/scrotum to permit proper ultrasound inspection. Secondly, and perhaps more importantly, particularly in situations where injury to the testes has occurred, the male patient is typically in a great deal of pain. The least movement and jostling of the testes/scrotum as possible is desired. Prior art towels frequently require frequent adjustment and positioning, thus unduly disturbing the testes/scrotum and causing the patient extreme discomfort.

In addition, due to the inherent flexibility of the towels, in some instances the towel(s) may undesirably cover a portion of the testes if not positioned correctly, thus resulting in increased movement to reposition the towels to conduct the ultrasound. They frequently, due to their lack of rigidity, permit the testes to move during ultasound examination, which is undesirable for the reasons aforesaid. Lastly, towels may possibly, due to their material, undesirably act to absorb some of the sound waves emitted by the ultrasound probe, thereby reducing the quality of the image obtained on the ultrasound monitor. Accordingly, towels do not serve as a good medium or base substrate for providing support to the testes/scrotum for an ultrasound examination.

### SUMMARY OF THE INVENTION

In order to overcome certain of the disadvantages of the prior art, the present invention in a broad aspect thereof provides for a simple device particularly suited and adapted to raise and support of male testes for purposes of permitting ultrasound examination thereof.

More specifically, the present invention in a broad aspect thereof comprises a device for raising and supporting human testes when upper legs of a patient are substantially horizontal, comprising:

a pair of spaced-apart, downwardly curved members, each adapted to be positioned over a respective upper leg of a patient and proximate a groin area of said patient; and

a support member situate intermediate said pair of curved members, adapted to raise and support one or more male testes when said device is positioned proximate said groin area.

In a second broad aspect of the invention, the invention comprises a device for raising and supporting one or more male testes of a patient during ultrasound examination when said patient is lying in a supine position, comprising:

a pair of spaced-apart, downwardly curved members, each adapted to be positioned on a respective upper leg of said patient; and

a support member, positioned intermediate said pair of curved members and coupled thereto, adapted to support one or more male testes thereon when positioned in a groin area of said patient.

In a third broad aspect of the present invention such invention comprises a device for supporting human testes when a male patient is lying in a supine position, comprising:

a support member, having mutually opposite side edges, adapted for positioning intermediate legs of said patient proximate a groin area of said patient to support one or more male testes of said patient; and

a downwardly curved member extending from each of said mutually opposite side edges of said support member, each adapted to rest upon a respective leg of said patient when said support member is positioned to support said testes.

It is preferred that the device be moulded in a unitary one-piece construction, of substantially rigid material, although two or three piece construction (support member with two curved members attached thereto on opposite side edges) is also contemplated.

The device is preferably of an acrylic, polystyrene, or polyethylene plastic, or other material impervious to water, to permit easily cleaning and sterilization after use, to permit re-use.

Plastic, such as moulded polystyrene, polyethylene, or acrylic is preferred as it permits moulding of the device in a one-piece construction which avoids the manufacturing step of attaching the curved members to the support member if a two or more piece construction is contemplated.

In a preferred embodiment, the device is a vacuum-formed acrylic plastic which is further transparent, although opaque or translucent materials may also be used.

Fabrication by stamping , such as in the case of the device is formed of a metallic material such as aluminium or stainless steel is also contemplated. However, due to the higher thermal conductivity of metals as opposed to plastics and the fact that such metals thus feel “cold” to the touch and thus are initially uncomfortable to the patient renders plastics a more suitable as a material.

Various sizes of device , varying for example in width of the support member and size and radius of curvature of the downwardly-curved members, are contemplated.

For example, a large size, having a relatively wide support member to provide adequate support to testes in a mature adult male, and having downwardly curved members each of a radius substantially equal to the radius of curvature of an mature adult male proximate the thigh area, are contemplated for use with adult males. Smaller sizes, with relatively narrow support members to permit positioning of such device in the groin area of male juveniles or smaller male children, having curved “wing” members of a correspondingly smaller radius of curvature to correspond to the radius of curvature of the leg of juveniles or small male children, are contemplated to permit use for smaller males.

Advantageously, due to the substantially rigid nature of the device, ultrasound technicians are able to substantially position the testes during repeat (subsequent) ultratound examination in the same position as the testes were positioned in one or more earlier ultrasound examination. This is hightly advantageous, in that it allows comparison between various examinations of the same patient, and progress (growth or reduction in size of a tumour, for example), is monitored over time. As well, measurements that are made of the testes via the images appearing on the monitor are able to be better compared to earlier or later measurements, due to the consistency in positioning of the testes when the device of the present invention is employed in each case.

### BRIEF DESCRIPTION OF THE DRAWINGS

Further advantages and permutations will appear from the following detailed description of various non-limiting embodiments of the invention, taken together with the accompanying drawings, in which:

FIG. 1 is a perspective view of a testicular support device of the present invention;

FIG. 2 is a perspective view of the device of FIG. 1, when positioned on a patient lying in a supine position; and

FIG. 3 is (rear) perspective view of the testicular support device shown in FIG. 1 taken in the direction of arrow “A”.

### DETAILED DESCRIPTION OF SOME PREFERRED EMBODIMENTS

In all figures, for consistency, identical components are identified with identical reference numerals.

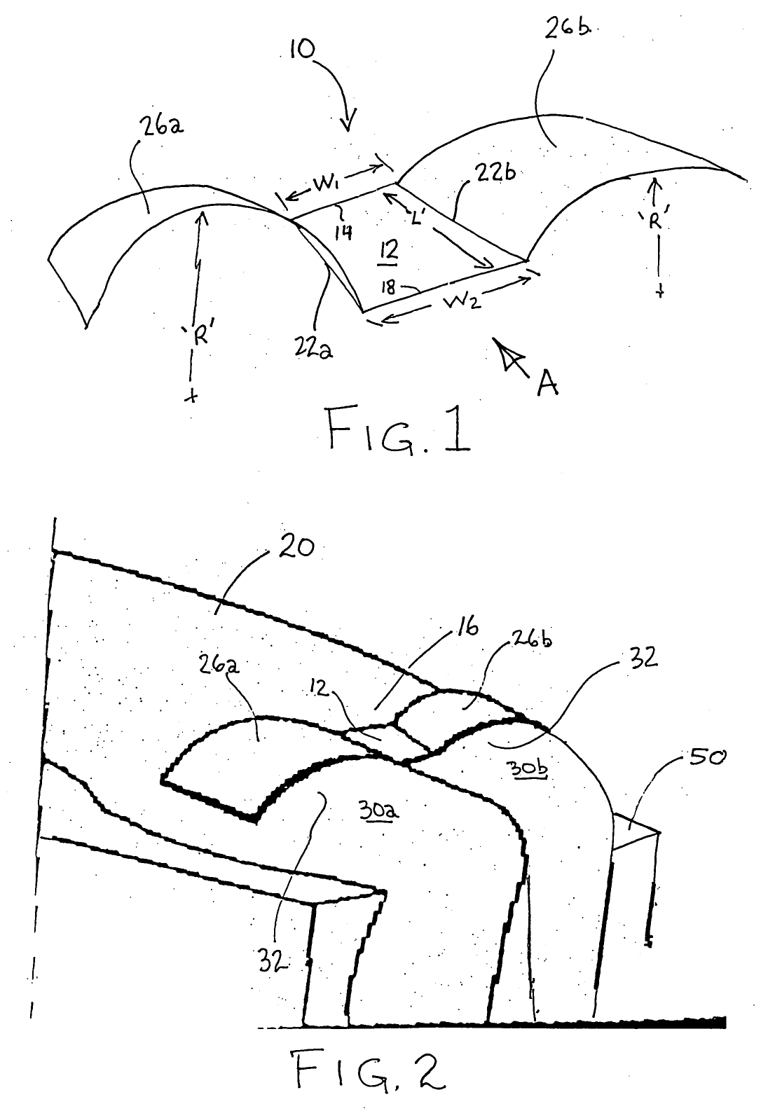

FIG. 1 shows a testicular support device 10 of the present invention, having support member 12, which in the embodiment shown is a substantially planar, increasing slightly in width , with the far side 14 thereof (the side preferably closest to a groin area 16 of a male patient 20) being slightly narrower in width than an opposite side 18.

Support member 12 is adapted to support one or more male testes (not shown) of male patient 20 and render them substantially immobile to permit ultrasonic inspection of said testes.

Extending from respective opposite side edges 22a, 22b of testicular support device 10 are downwardly-curved members 26a, 26b, each adapted to be positioned over a respective leg 30a, 30b of male patient 20 (see FIG. 2) when such device 10 is positioned in groin area 16 of male patient 20. Downwardly-curved members 26a, 26b effectively suspend support member 12 therefrom, so as to permit such support member 12 to support the testes of patient 12, when such downwardly-curved members 26a, 26b are positioned on the thighs 32 of male patient 20.

Downwardly curved members 26a, 26b are preferably curved and preferably, although not necessarily, have a circular or substantially circular radius “R” approximating equal to the radius of a patient's leg 30a,30b proximate the thigh 32 of said patient 20. As most humans legs gradually and uniformly decrease in radius from the groin area downwardly toward the foot(not shown) of patient 20, such curved members 26a, 26b may likewise gradually and uniformly decrease in curvature over the length “L” of device 10, in order that the downwardly curved members 26a, 26b better rest on respective legs 30a, 30b of patient 20.

Preferrably, device 10 is of various sizes. In particular, for adult males, the radius R, and widths W₁ and W₂ of support member 12 are greater than corresponding dimensions of a testicular support device 10 adapted for smaller adults, juveniles, or children.

FIG. 2 shows the device 10 properly positioned for use on a patient 20, when such patient 20 is lying in a substantially supine position on an examination table 50, with the upper leg /thighs 32 of said patient 20 substantially horizontal. Downwardly curved members 26a, 26b are each positioned so as to rest on upper thighs 32 of respective legs 30a, 30b, so as to support support member 12 which in turn supports and immobilizes testes (not shown) of patient 20.

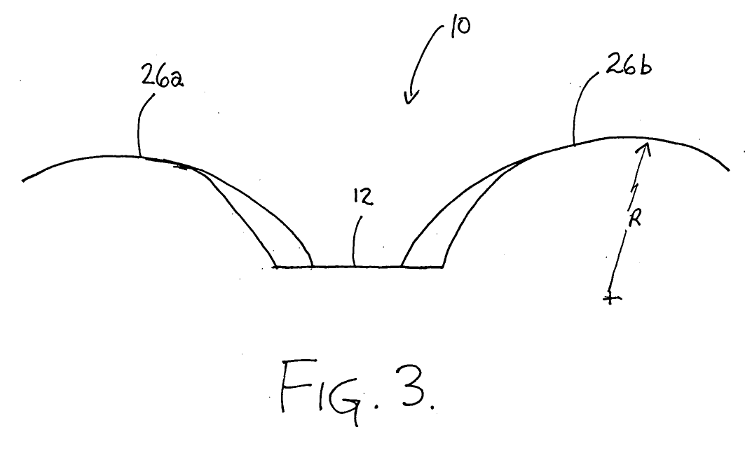

FIG. 3 shows an end view of the testicular support device 10 taken in the direction of arrow “A” of FIG. 1. In the embodiment shown, radius “R” of each of downwardly curved members 26a, 26b is progressively reduced over the length “L” of the device 10 (progressing from front to rear of device 10) , so as to better conform and rest on the legs 30a, 30b of a patient 20 and to better allow the support member 12 to remain stationary and thereby maintain the testes which it supports immobile to permit ultrasonic examination thereof.

However, such radius “R” may be fixed so as to maintain a fixed curvature of each of the downwardly curved members 26a, 26b over length “L” of device 10, if desired.

Although the disclosure described and illustrates preferred embodiments of the invention, it is to be understood that the invention is not limited to these particular embodiments. Many variations and modifications will now occur to those skilled in the art. For definition of the invention, reference is to be made to the appended claims.

I claim:

1. A device for raising and supporting human testes when upper legs of a patient are substantially horizontal, comprising:
a pair of spaced-apart, downwardly curved members, each adapted to be positioned over a respective upper leg of a patient and proximate a groin area of said patient; and
a support member situate intermediate said pair of curved members, adapted to raise and support one or more male testes when said device is positioned proximate said groin area.

2. A device for raising and supporting one or more male testes of a patient during ultrasound examination when said patient is lying in a substantially supine position, comprising:
a pair of spaced-apart, downwardly curved members, each adapted to be positioned on a respective upper leg of said patient; and
a support member, positioned intermediate said pair of curved members and coupled thereto, adapted to support one or more male testes thereon when positioned in a groin area of said patient.

3. A device for supporting human testes when upper legs of a patient are substantially horizontal, comprising:
a support member, having mutually opposite side edges, adapted for positioning intermediate legs of said patient proximate a groin area of said patient to support one or more male testes of said patient; and
a downwardly curved member extending from each of said mutually opposite side edges of said support member, each adapted to rest upon a respective leg of said patient when said support member is positioned to support said testes.
